(19) United States
(12) Patent Application Publication
(10) Pub. No.: US 2014/0318222 A1
(43) Pub. Date: Oct. 30, 2014

(54) METHOD TO PREVENT EXCESSIVE MAINTENANCE TO A VEHICLE LUBRICATING SYSTEM BASED ON DETECTION

(71) Applicant: MIT AUTOMOBILE SERVICE COMPANY LIMITED, YANTAI (CN)

(72) Inventors: Zhixin XIAO, YANTAI (CN); Qin WEI, YANTAI (CN); Zhiliang WEI, YANTAI (CN); Qinglu CUI, YANTAI (CN)

(73) Assignee: MIT AUTOMOBILE SERVICE COMPANY LIMITED, YANTAI (CN)

(21) Appl. No.: 14/264,050

(22) Filed: Apr. 28, 2014

(30) Foreign Application Priority Data
Apr. 27, 2013 (CN) 201310152465.4

(51) Int. Cl. G01N 33/28

(52) U.S. Cl. CPC G01N 33/2888
USPC 73/53.05

(57) ABSTRACT

This invention is a method to prevent excessive maintenance to vehicle lubricating system based on detection, to overcome the defect in maintenance procedure. Technical measures are as follows: it is a method to prevent excessive maintenance to vehicle lubricating system based on detection, with oil detection module 1 and host controller 4. Its working principle is to detect the aging degree of lube before maintenance, and decide whether to change the lube according to detection result. It will help to prevent excessive maintenance, reduce maintenance cost for car owners, and decrease the waste of resource. Oil detection module 1 includes oil chamber sensor 2, capacitance detection chip 3 etc. Host controller 4 is connected to oil detection module 1 by cables.

### FIELD OF THE INVENTION

This invention is a method to prevent excessive maintenance to vehicle lubricating system based on detection.

### BACKGROUND

Maintenance to lubricating system is the most important part of car maintenance. A bad lubrication to motor will increase fuel consumption and abrasions to motor, at the same time, shorten lifespan of motor. At present, we maintain the lubricating system according to interval of service time or travel distance, and ignore detection to aging degree of engine lubricating oil. This excessive maintenance increase customers' economical load and result in wasting of resources.

### SUMMARY OF THE INVENTION

This invention is to overcome the defect in maintenance to car lubricating system.

Technical measures are as followings:

It is a method to prevent excessive maintenance to vehicle lubricating system based on detection, with oil detection module and host controller. Its working principle is to detect the aging degree of lube before maintenance, and decide whether to change the lube according to detection result. It will help to prevent excessive maintenance, reduce maintenance cost for car owners, and decrease the waste of resource.

Oil detection module includes oil chamber sensor, capacitance detection chip, and etc. Host controller is connected to oil detection module by cables.

Specific methods:

1. The systems includes oil detection module, host controller, and etc. Oil detection module includes oil chamber sensor, capacitance detection chip etc. Host controller is connected to oil detection module by cables. Host controller includes main control board, barcode scanning module, LCD touch screen, electric pump, electromagnetic valve, and coupling. Main control board is connected to barcode scanning module, LCD touch screen, electric pump, electromagnetic valve by cables. Coupling is connected to electric pump, electromagnetic valve by tubes.

2. Characteristic: Before maintenance, detect engine lubricating oil with oil detecting module. Contrast and calculate the variation of mass fraction of water, iron content, and acid value with normal data. Decide whether to change lubricating oil according to aging degree in detection result.

This invention has simple and convenient operation to prevent excessive maintenance and wasting of resources.

### BRIEF DESCRIPTION OF THE DRAWINGS

FIG. 1 is system principle diagram.

FIG. 2 is flow diagram of rapid oil detection.

FIG. 3 is flow diagram of standard oil detection.

The following call out list of elements can be a useful guide in referencing the elements of the drawings.
1 oil detection module
2 oil chamber sensor
3 capacitance detection chip
4 host controller
5 control board
6 electric pump
7 electromagnetic valve
8 LCD touch-screen
9 barcode scanning module
10 coupling

### DETAILED DESCRIPTION OF THE PREFERRED EMBODIMENT

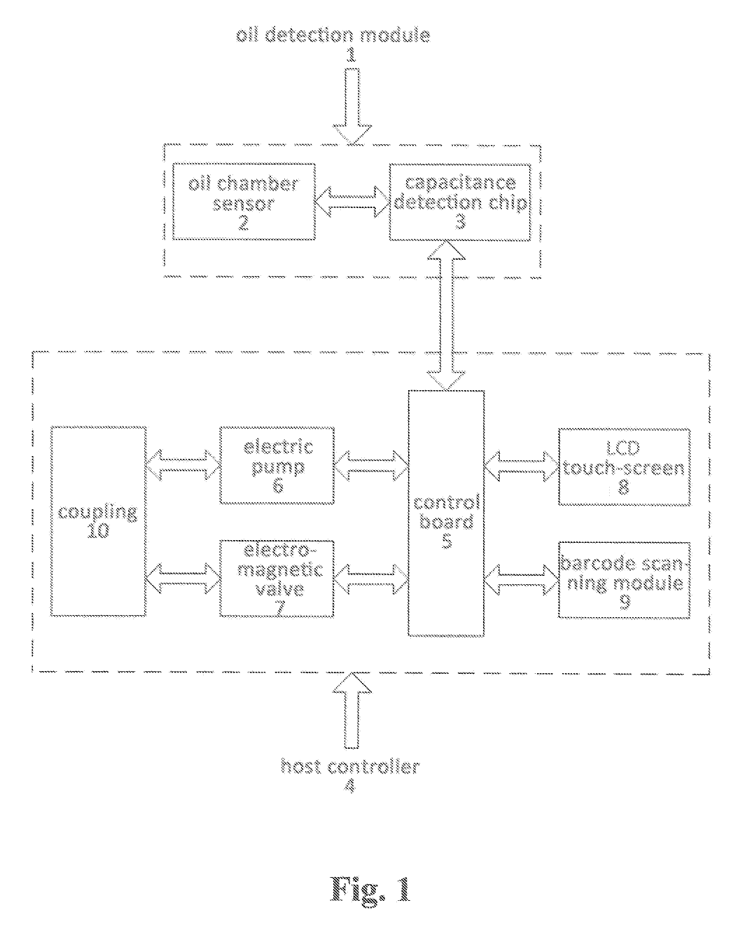

Please refer to FIG. 1 for system principle diagram. 1. System includes oil detection module 1 and host controller 4.

Oil detection module 1 includes oil chamber sensor 2, capacitance detection chip 3. Oil chamber sensor 2 is connected to capacitance detection chip 3 by connector.

Host controller 4 includes main control board 5, barcode scanning module 9, LCD touch-screen 8, electric pump 6, electromagnetic valve 7 and coupling 10. Main control board 5 is connected to barcode scanning module 9, LCD touch-screen 8, electric pump 6, and electromagnetic valve 7 by cables. Coupling 10 is connected to electric pump 6, electromagnetic valve 7 by tubes.

Oil detection module is connected to host controller by cables.

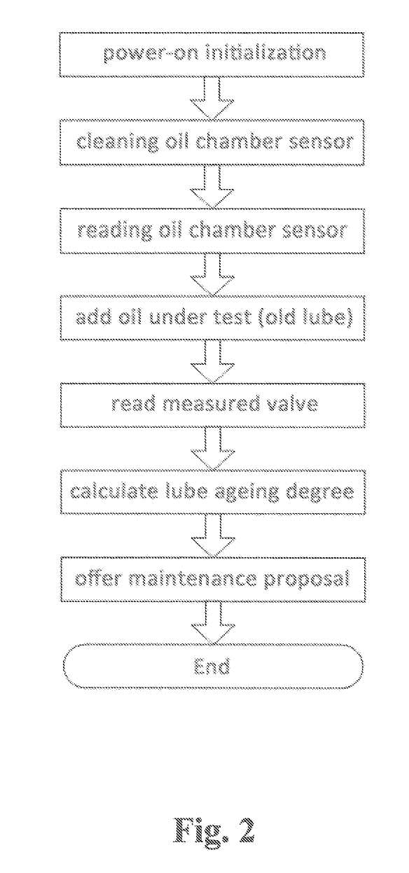

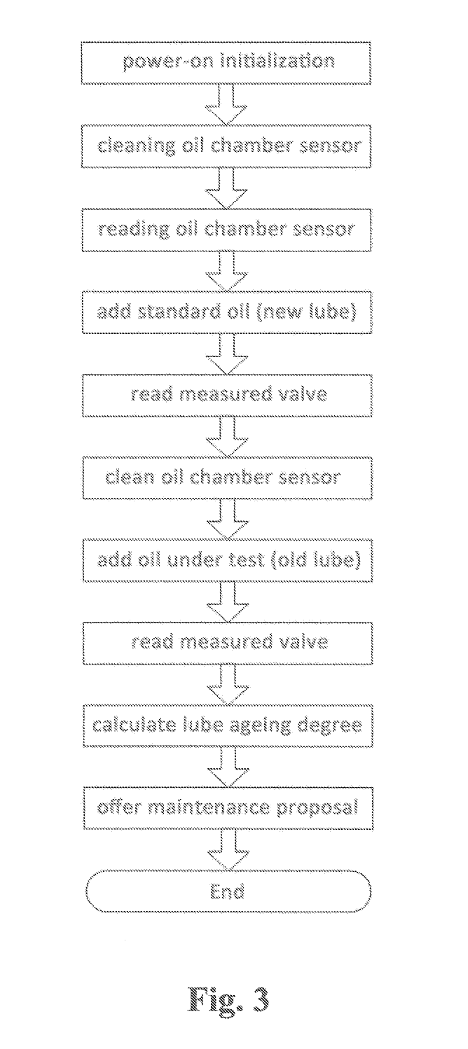

Please refer to FIG. 2 and FIG. 3 for rapid and standard oil detection method. Add oil sample to oil chamber sensor 2 when measuring. Measured data will be read by main control board 5 through capacitance detection chip 3.

In FIG. 2, after power on initialization, clean oil chamber sensor 2, and read its valve without oil. Add oil under test old oil to oil chamber sensor 2, and read measured value. Contrast and calculate measured valve with standard value to gain aging degree of lubricating oil. If measured valve is higher, change lube; if not, do not change it.

In FIG. 3, after power on initialization, clean oil chamber sensor 2, and read its valve without oil. Add standard oil new lube to oil chamber sensor 2, and read measured valve. Re-clean oil chamber sensor 2, add oil under test old oil to oil chamber sensor 2, and read measured value. Contrast and calculate measured valve with standard value to gain aging degree of lubricating oil. If measured valve is higher, change lube; if not, do not change it.

This invention detects parametric of lubricating oil for its aging degree by rapid/standard oil detection method to offer suggestion to maintenance of lubricating system. This invention has simple and convenient operation to prevent excessive maintenance and wasting of resources.

1. It is a method to prevent excessive maintenance to vehicle lubricating system based on detection, with oil detection module 1 and host controller 4. Its working principle is:
detect the aging degree of lube before maintenance, and decide whether to change the lube according to detection result. It will help to prevent excessive maintenance, reduce maintenance cost for car owners, and decrease the waste of resource.

2. Oil detection module 1 includes oil chamber sensor 2, capacitance detection chip 3 etc;
host controller 4 includes main control board 5, barcode scanning module 9, LCD touch-screen 8, electric pump 6, electromagnetic valve 7 and coupling 10. Host controller 4 is connected to oil detection module 1 by cables.
